(19) United States
(12) Patent Application Publication
(10) Pub. No.: US 2005/0137532 A1
(43) Pub. Date: Jun. 23, 2005

(54) Unit to administer injectable medication manually or automatically

(76) Inventor: Jose Santiago Rolla, Buenos Aires (AR)

Correspondence Address: Paul L. Brown, Esq.;EMRICH & DITHMAR LLC, Suite 2080, 125 South Wacker Drive, Chicago, IL, 60606, US

(21) Appl. No.: 10/833,794

(22) Filed: Apr. 28, 2004

(30) Foreign Application Priority Data
Dec. 18, 2003 (AR) P030104719

(51) Int. Cl. A61M 5/315

(52) U.S. Cl. 604/218

(57) ABSTRACT

This injecting unit (1) comprises a tubular body (2) with a receptacle (6) containing a solution; this receptacle (6) is delimited by an impelling plunger (4) and a drillable bottom (5); the drillable bottom (5) faces a perforating means (7) communicated with an injection nozzle (3); the impelling plunger (4) can be connected to automatic or manual action rods (10). Therefore, the injecting unit (1) may be used both in machines and devices [dispensers, fractionators, mixers, dosing apparatus] and manually. The rod (10) may include stroke stops (14) which allow dispensing medication in doses.

### I—BACKGROUND OF THE INVENTION

1. Field of Invention

The present invention is related to the field of means and devices for the application of injections. It is a unit to administer injectable medication manually or automatically which can be used both manually and automatically within devices and machines.

2. Description of Prior Art

Medication is injected by means of syringes in which generally the medication to be injected must be drawn from vials. This operation is done manually, which, besides being arduous and slow, frequently causes the contamination of the medication basically due to its contact with ambient air, and which often presents other technical deficiencies.

Generally, all the systems and methods of administering medication have not been adapted to the evolution experienced by the medical and welfare technologies. At present, more than 80% of the world injectable medication is given in a personalized and scheduled manner, in the case of inpatients, and it is administered intravenously for periods which vary according to the patient's status and the doctor's criterion. This routine also consists in drawing the medication manually from the vials into the injection syringes, and from there into the containers of sterile solutions for intravenous use. These manual procedures used in conventional syringes with the medication to be administered are carried out in non-sterile environments and, besides being very slow and complex, they are technically unsafe and involve a high risk due to the danger of the possibility of contamination because the injectable medication is in contact with non-sterile ambient air, and other technical faults such as possible mistakes in the dosage or incorrect identification of the medication or of the patients to whom it should be given. As a result of this deficient technology, frequent complications arise which worsen the patients status, which is generally followed by lawsuits for malpractice against medical institutions and doctors. Besides these serious inconveniences, these methods involve complicated, slow and inefficient administrative controls of the used and stored medication and of their invoicing processes. As can be clearly inferred, with this technology for drawing medication under conditions with no guaranteed asepsis, no medical institution of the world is ready to comply with ISO quality control standards as regards controls and methodology related to the administration of injectable medication.

From the point of view of ecology, with current systems and due to the dispersion of the elements used, consideration should be given to the environmental contamination caused by syringes, their needles and protective caps, glass vials and used flexible plastic containers, and to another serious risk of contamination implied by the possibility of reusing disposable syringes. Furthermore, the risk to which health care workers and all the staff handling medical refuse are exposed is very serious, due to the possibility of suffering accidental needle stick injuries occurring with needles contaminated with AIDS or other serious infectious diseases which inpatients might suffer and which are frequent in medical institutions.

There are various well known manual, automatic or semi-automatic systems of injecting medication in a single or multiple way, and syringes aimed at optimizing the application and transfer of injectable medication, such as those disclosed in the following patents: U.S. Pat. No. 3,670,923, U.S. Pat. No. 4,111,335, U.S. Pat. No. 4,333,356 U.S. Pat. No. 4,467,844, U.S. Pat. No. 4,509,861, U.S. Pat. Nos. 4,513,796; 4,527,245; U.S. Pat. No. 4,625,494, U.S. Pat. No. 5,431,201, UK GB 2,061,747; PCT/SE97/01094, EP 0 335 378 A2; EP 0 882 441 A2, EP 0 898 951 A2, U.S. Pat. No. 0,003,566; U.S. Pat. No. 6,039,093, and PCT/SE 00/01445. For more than fifty years, odontologists all over the world have been using a system which contains injectable anesthetic in a tubular element within a space determined between two plungers, but a special syringe with a piston and a perforation system of the lower plunger is needed to inject its content. However, in the present invention, the injecting unit autonomously and permanently contains the plunger perforation system and the medication injection or transfer system. The patents and systems mentioned and various others develop multiple syringes and systems to mix and administer automatically or semi-automatically injectable or oral solutions, but since they fail to provide satisfactory solutions to current needs, none of them has been implemented and made known massively.

### II—SUMMARY OF THE INVENTION

The present invention comprises a unit to administer injectable medication manually or automatically, with a tubular body inside of which there is a receptacle delimited by a solution impelling plunger and by a drillable bottom, on which a perforating means actuates and which communicates with the outside part of the receptacle through the injection nozzle.

Objects and Advantages.

The present invention efficiently solves all these inconveniences, thus beginning the first stage of a new technology related to the safe administration of injectable medication subcutaneously, without being drawn in and out, thus guaranteeing absolute sterility. The system's versatility allows to begin the first stage of a new intravenous medication application system, providing the possibility of automatically bottling, in an absolutely sterile system, the injectable medication scheduled in a personalized manner, keeping its sterility and avoiding the possibility of any kind of mistake, by means of electronic controls using bar code reading sensors and the like, which control all the steps in the transfer of injectable medication into the containers of intravenous solutions. To achieve this, the present invention provides manual or automatic injection of medication by means of new elements which constitute “Injecting Units” (IUs), in which the contained liquid medication and the thin tubular element from which the injectable medication flows are bottled jointly under sterility conditions during the manufacturing process, and which due to their special design and internal devices are excellent for these and other purposes.

The IUs consist in practical and efficient devices which practically, safely and efficiently replace the joint function of vials and syringes currently used, allowing the direct fast injection of medication manually or through automatic systems, inside the containers of intravenous solutions. Each one of them may contain one or multiple doses of injectable medication.

In the case of emergency injectable medication of non-scheduled application in which the injection must be administered through elastomer diaphragms of the tubular elements of intravenous injection, which regulate the flow of medication, the present invention allows to do this using the element which enables the manipulation of the (IU), through a manufacturing variant wherein the lower end of the tubular element along which the injectable medication is transferred ends in a sharp pointed tip which, by means of enough pressure exerted manually using a piston, perforates the elastomer body to allow the flow of the injectable medication.

As it can be appreciated, this system provides speed and absolute safety in the administration of injectable medication, minimizing the possibility of human mistakes involved in the manual transfer, avoiding contamination of the medication by being in contact with ambient air and due to technical failures, and the mistakes in the medication administration, since on the surface of the (IUs) the name is printed using letters that can be read with the naked eye or bar codes or other automatic reading systems of identification, as well as inscriptions which, by being read, allow to know the place and position of the (IUs) so as to be used in the automatic system.

As regards environment and environmental care, this invention makes it impossible for health care workers and the staff handling medical refuse to suffer accidental injuries or other types of damages caused by contaminated needles. Moreover, it prevents the injecting unit from being reused, since it immediately becomes useless after being used, and it allows for the absolute joint recycling of glass and plastic materials, elastomer and any other material used in the manufacture of the injecting units.

Another important advantage of the present invention is that by joining in a single element the ones that are currently used separately, it simplifies and makes the industrial bottling processes of injectable medication cheaper, by reducing the amounts of materials used in the manufacturing process and, thus, storing and transport spaces and costs.

This invention reduces the chances of human or other kinds of mistakes in the injectable medication application process, in every moment of the manual injection process or the programmed and personalized automatic transfer and bottling process, further preventing the injectable medication from being in direct contact with non-sterile ambient air, which constitutes an important contaminating factor.

The Injecting Units may also contain amino acids, liquid foods and other sterilized compounds to be transferred and mixed into sterile containers to be orally, injectably or otherwise administered. They are ideal to prepare doses and mix solutions used for renal dialysis. In other types of applications, they are suitable to contain and transfer any type of fluid or viscous substance difficult to handle and that should be mixed.

The (IUs) allow to inject non-scheduled emergency medication through the elastomer membranes of tubular systems of intravenous application. They are also ideal for the application of vaccines or other medication dispensed subcutaneously. Furthermore, the contained injectable medication can be transferred under absolute sterility conditions into the usual disposable syringes, to be injected conventionally, by intramuscular or direct intravenous injection.

### III—DESCRIPTION OF THE DRAWINGS

For the sake of clarity and to understand the object of the invention, it is illustrated in different figures in which it has been represented in one of the preferred embodiments, which is purely exemplary and therefore nonlimiting:

FIG. 1 is a longitudinal view of the injecting unit in which the perforating means consists of an inner needle which forms an integral part of the tubular body (2), being fixed to the formed end wall (3a) of the injection end (2b) in an embodiment suitable for automatic or manual use.

FIG. 2 is a longitudinal view of another embodiment suitable for automatic or manual use in which the perforating means consists of a hollow needle which forms integral part of the fixed end wall (3b) to the injection end (2b) of the tubular body (2).

FIG. 3 comprises drawings A, B and C, wherein: drawing A is a longitudinal view showing the forward movement of an automatic action rod; drawing B is an elevation top view of the fixable part in which the perforating means and the injection nozzle are situated; and drawing C is an elevation side view of the part in drawing B.

FIG. 4 comprises drawings A, B and C, wherein: drawing A is a perspective view of a removable cap with elastic clamp; drawing B is a perspective view of a removable cap without elastic clamp; and drawing C is a perspective view showing the application of the cap to the injection nozzle.

FIG. 5 is a longitudinal view of the tubular body with the applied cap.

FIG. 6 comprises drawings A, B and C, wherein: drawing A is a perspective view of a rod provided with stroke stops for dosing and an impelling plunger provided with coupling means to the rod; drawing B is a cross-sectional view of the elongated body of the rod according to section lines indicated in drawing A; and drawing C is a cross-sectional view of the impelling plunger showing the coupling means to the rod.

FIG. 7 is a longitudinal view of a manual application set including the injecting unit, the additional support and an injection needle.

FIG. 8 is another longitudinal view showing how the stroke stops are used.

FIG. 9 is another longitudinal view showing the injection needle arrangement.

FIG. 10 comprises drawings A, B and C, wherein: drawing A is a longitudinal view of a plunger (in the case of a drillable bottom, it would be similar) wherein the sealing means have a rounded projecting section; drawing B is another longitudinal view wherein the sealing means have a rectangular projecting section; and drawing C is another longitudinal view wherein the sealing means have an angular projecting section.

FIG. 11 is a perspective view illustrating the arrangement of the injecting unit and its needle in an additional support.

FIG. 12 is another perspective view as the one in FIG. 11, but in which the rod has been added, thus forming the set ready for manual use.

FIG. 13 comprises drawings A and B, wherein: drawing A is a perspective view of the fitting recess of the additional support; and drawing B is another perspective view in which the needle head is fitted in the recess.

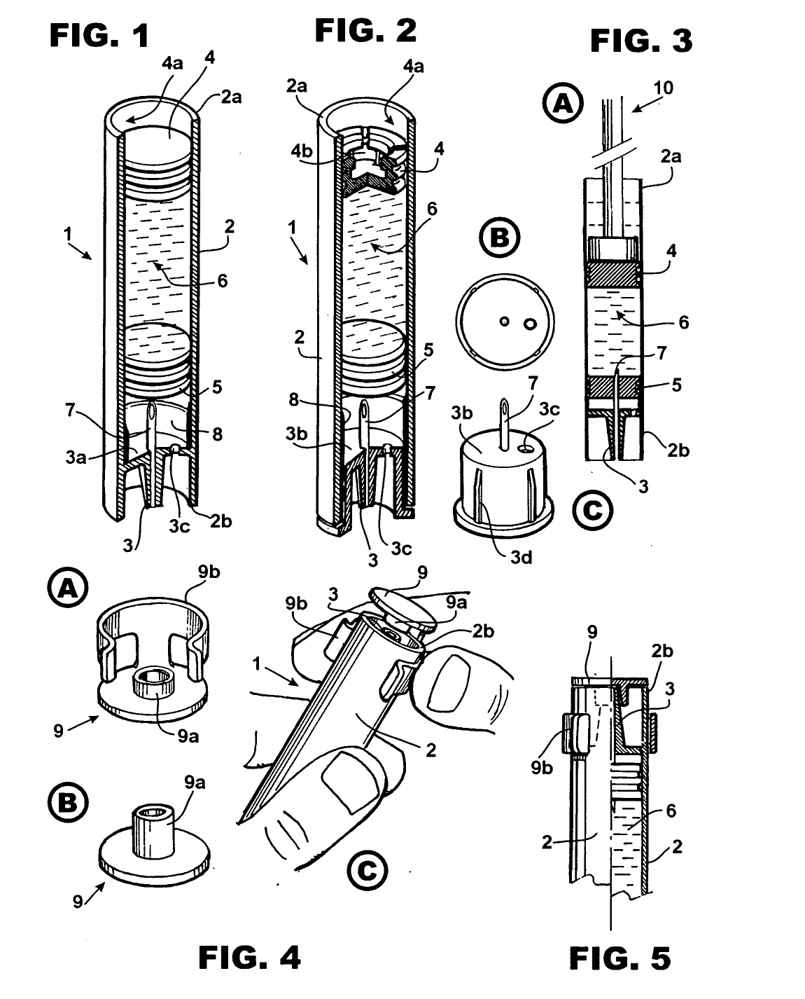

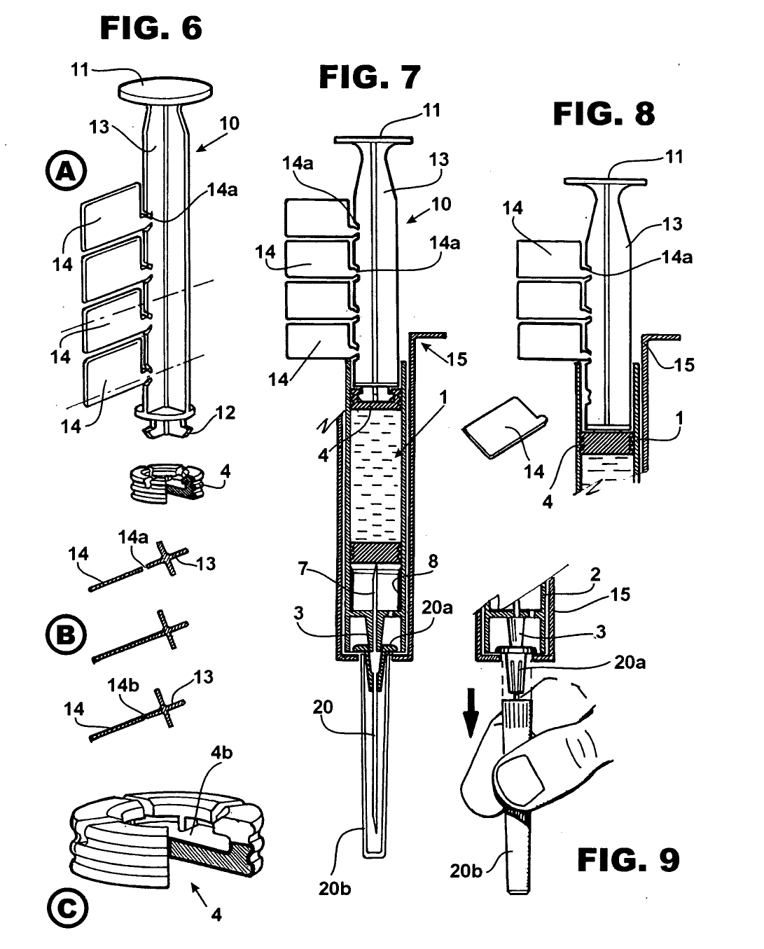

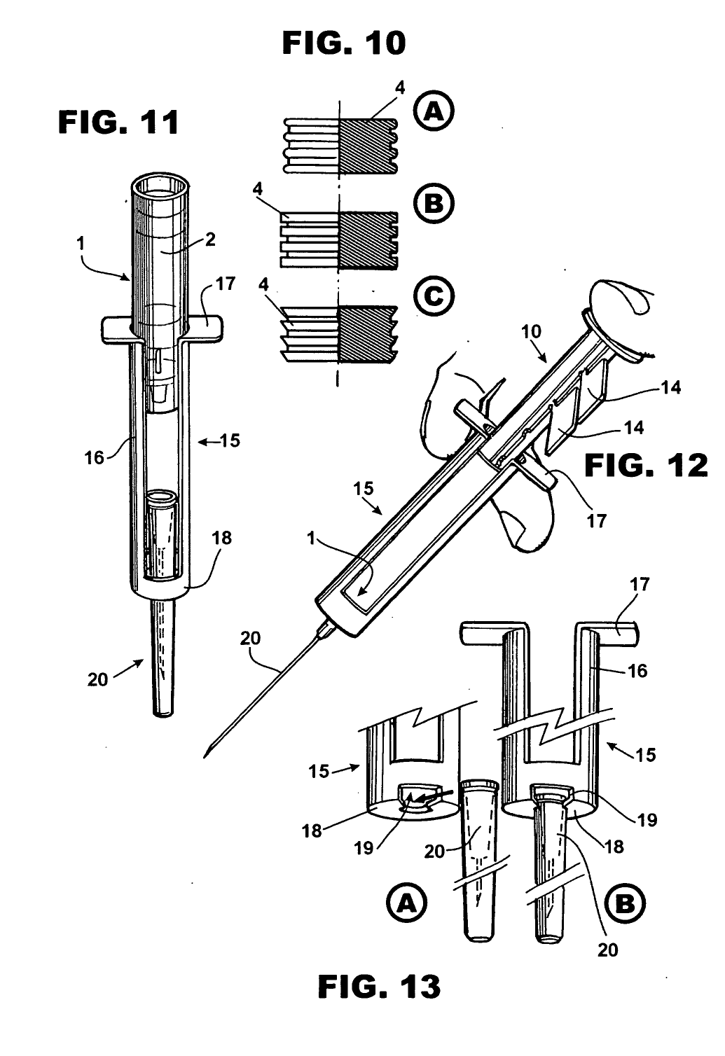

In the different figures, similar reference numbers and/or letters designate similar or corresponding parts.

Reference Numerals in Drawings:

(1) Injecting unit.
(2) Tubular body.
(2a) Command end of the tubular body (2).
(2b) Injection end of the tubular body (2).
(3) Injection nozzle.
(3a) Formed end wall.
(3b) Fixed end wall.
(3c) Air outlet.
(3d) Fixing means of the fixed end wall (3b).
(4) Impelling plunger.
(4a) Guiding cavity.
(4b) Coupling means to the rod (10).
(5) Drillable bottom.
(6) Receptacle for medicinal solution.
(7) Perforating means.
(8) Retentive means of the drillable bottom (5).
(9) Removable cap.
(9a) Mouth for the injection nozzle (3).
(9b) Elastic clamp.
(10) Rod.
(11) Rod lug (10).
(12) Coupling means to the impelling plunger (4).
(13) Elongated body of the rod (10).
(14) Nonreusable stroke stops [dosing means].
(14a) Cutting area of the stroke stops (14).
(14b) Weakening lines in the cutting areas (14a).
(15) Additional support.
(16) Branching walls of the additional support (15).
(17) Side lugs of the additional support (15).
(18) Injection end of the additional support (15).
(19) Needle fitting recess (20).
(20) Injection needle.
(20a) Needle head (20)
(20b) Needle protective cap (20).

### IV—DETAILED DESCRIPTION

The present invention consists in a unit to administer injectable medication manually or automatically which, in general terms, comprises a tubular body (2) with a receptacle (6) containing a solution, said receptacle (6) is delimited by an impelling plunger (4) and a drillable bottom (5); the drillable bottom (5) faces a perforating means (7) communicated with an injection nozzle (3); the impelling plunger (4) can be connected to manual or automatic action rods (10).

More particularly, the present unit (1) to administer injectable medication can be used both manually or in machines or devices used for automatically dosing, mixing or dispensing injectable solutions. Each unit (1) contains a medicinal solution which can be applied in one or more doses, and it is disposable once said solution has been completely applied.

The present injecting unit (1) comprises a tubular body (2) which on one end finishes in a command end (2a) and on the opposite end finishes in an injection end (2b).

Inside the tubular body (2) there is an impelling plunger (4) and a drillable bottom (5), between which a receptacle (6) containing the injectable solution is defined.

Said drillable bottom (5) faces a perforating means (7) located at an end wall (3), from which an injection nozzle (7) is formed. Inside there is a passage communicating the perforating means (7) with said injection nozzle (3). Furthermore, there is an air outlet (3c) which allows the drillable bottom (5) to move towards the perforating means (7).

The perforating means (7) may consist of an internal needle or a tubular element thicker than the sharp end.

The addition of retentive means (8) of the drillable bottom (5) has been provided. These retentive means (8) may consist in a sectional narrowing of the tubular body (2) so that the drillable bottom (5) may move forwards but not backwards.

The injection nozzle (3) is sunk within the limits of the tubular body (2) walls, without projecting with respect to the injection end (2b).

The end stop wall (3a)(3b) in which the perforating means (7) and the injection nozzle (3) are located may be formed (3a) by the tubular body (2) proper, or it may consist in a part fixed (3b) by fixing means (3d) to the injection end (2b).

The injection nozzle (3) is prepared to be connected to needles (20), cannulas, tubes or any other equivalent element.

A removable cap (9) has also been provided. This cap (9) has a funnel (9a) in which the nozzle (3) is fitted, and a resilient clamp (9b) which allows to fix it to the tubular body (2).

Furthermore, the impelling plunger (4) is slightly away from the command end (2a) so that a guiding cavity (4a) is formed through which the command rod (10) enters.

The present injecting unit (1) is suitable for use both with automatic action rods (10) which are part of machines or devices and manual action rods (10).

In the case of manual use, the impelling plunger (4) may have coupling means (4b) [for example, notched-grooved] which allow the connection with corresponding coupling means (12) provided by the rod (10).

It has also been provided that the elongated body (13) of the rod (10) has nonreusable stroke stops (14) as dosing means. Thus, there may be plural nonreusable stroke stops (14) sequentially arranged along the elongated body (13), so that each used stroke stop (14) enables the forward movement of the impelling plunger (4) and the administration of a corresponding dose. Preferably, the stroke stops (14) will have weakening lines (14b) in the cutting areas (14a), which will make their removal easier.

The use of an additional support (15) has also been provided to accommodate the injecting unit (1). This additional support (15) is formed by branching walls (16) forming longitudinal openings through which the injecting unit (1) is exposed. The branching walls (16) end in two side lugs (17) and, in the opposite end, they converge into an injection end (18). Said injection end (18) has a recess (19) suitable for the head (20a) of a needle (20) to fit in.

Both the impelling plunger (4) and the drillable bottom (5) have sealing means which act against the tubular walls (2) of the injecting unit (1). These sealing means consist of an annular grooving, the ledges of which may be of rounded, rectangular or angular section, the sloping side of which is arranged so that it helps with the forward movement inside the tubular body (2).

1) UNIT TO ADMINISTER INJECTABLE MEDICATION MANUALLY OR AUTOMATICALLY; which, being suitable for both manual use and for use in machines or devices, may contain a medicinal solution to be administered in one or more doses, and which is disposable once said solution has been completely used, having:
A tubular body which, between a command end and an injection nozzle, forms a receptable which contains the medicinal solution;
and comprising:
On its opposing ends, the receptacle is delimited by a plunger impelling the solution and a drillable bottom;
Said impelling plunger may be connected to manual or automatic action rods;
Inside the injection nozzle there is a perforating means which faces said drillable bottom; and
Said perforating means communicates with the outside of the receptacle through the injection nozzle.

2) UNIT TO ADMINISTER INJECTABLE MEDICATION MANUALLY OR AUTOMATICALLY; according to claim 1; wherein the plunger has coupling means to the end of a command rod.

3) UNIT TO ADMINISTER INJECTABLE MEDICATION MANUALLY OR AUTOMATICALLY; according to claim 2; wherein between the plunger and the end of the command rod there are notched-grooved coupling means.

4) UNIT TO ADMINISTER INJECTABLE MEDICATION MANUALLY OR AUTOMATICALLY; according to claim 1; wherein between the command end of the receptacle and the plunger there is a guiding cavity defined for a command rod.

5) UNIT TO ADMINISTER INJECTABLE MEDICATION MANUALLY OR AUTOMATICALLY; according to claim 1; wherein the perforating means is a needle.

6) UNIT TO ADMINISTER INJECTABLE MEDICATION MANUALLY OR AUTOMATICALLY; according to claim 1; which around the perforating means, the receptacle has retentive means of the drillable bottom so that it allows the movement towards the nozzle and prevents the movement backwards.

7) UNIT TO ADMINISTER INJECTABLE MEDICATION MANUALLY OR AUTOMATICALLY; according to claim 1; wherein adjacent to the nozzle there is an air outlet of the receptacle which communicates the outside to the variable cavity formed between the drillable bottom and said nozzle.

8) UNIT TO ADMINISTER INJECTABLE MEDICATION MANUALLY OR AUTOMATICALLY; according to claim 1; wherein the nozzle is sunk within the limits of the tubular body walls, without projecting with respect to the injection end of said tubular body.

9) UNIT TO ADMINISTER INJECTABLE MEDICATION MANUALLY OR AUTOMATICALLY; according to claim 1; wherein the perforating means and the injection nozzle are on a stop end wall which is the advance limit of the drillable wall inside the receptacle.

10) UNIT TO ADMINISTER INJECTABLE MEDICATION MANUALLY OR AUTOMATICALLY; according to claim 9; wherein the stop end wall is formed by the tubular body.

11) UNIT TO ADMINISTER INJECTABLE MEDICATION MANUALLY OR AUTOMATICALLY; according to claim 9; wherein the stop end wall is fixed to the injection end of the tubular body.

12) UNIT TO ADMINISTER INJECTABLE MEDICATION MANUALLY OR AUTOMATICALLY; according to claim 1; characterized in that the nozzle is prepared to be connected to needles, cannulas, tubes and the like.

13) UNIT TO ADMINISTER INJECTABLE MEDICATION MANUALLY OR AUTOMATICALLY; according to claim 1; wherein the nozzle has a removable cap.

14) UNIT TO ADMINISTER INJECTABLE MEDICATION MANUALLY OR AUTOMATICALLY; according to claim 13; wherein the cap fits in the nozzle and has a resilient clamp as a fixing means to the end adjacent to the tubular body.

15) UNIT TO ADMINISTER INJECTABLE MEDICATION MANUALLY OR AUTOMATICALLY; according to claim 1; wherein the command rod has, at least, one nonreusable stroke stop as a dosing means.

16) UNIT TO ADMINISTER INJECTABLE MEDICATION MANUALLY OR AUTOMATICALLY; according to claim 1; wherein the command rod has plural nonreusable stroke stops, sequentially arranged along said rod.

17) UNIT TO ADMINISTER INJECTABLE MEDICATION MANUALLY OR AUTOMATICALLY; according to claim 15; wherein the stroke stops have respective weakening lines that determine the cutting area and removal with respect to the body of the command rod.

18) UNIT TO ADMINISTER INJECTABLE MEDICATION MANUALLY OR AUTOMATICALLY; according to claim 1; which has an additional support which accommodates the tubular body and which, on one end, forms a handgrip means while, on the opposite end, forms an injection end against which the nozzle is arranged.

19) UNIT TO ADMINISTER INJECTABLE MEDICATION MANUALLY OR AUTOMATICALLY; according to claim 18; wherein the injection end, against which the nozzle is arranged, forms a recess prepared for the head of a needle to fit in.

20) UNIT TO ADMINISTER INJECTABLE MEDICATION MANUALLY OR AUTOMATICALLY; according to claim 18; wherein the additional support forms a housing open lengthwise for the tubular body; the longitudinal openings are delimited by branching walls which start at the injection end and finish in two lugs.

21) UNIT TO ADMINISTER INJECTABLE MEDICATION MANUALLY OR AUTOMATICALLY; according to claim 1; wherein the impelling plunger has sealing means consisting of an annular grooving.

22) UNIT TO ADMINISTER INJECTABLE MEDICATION MANUALLY OR AUTOMATICALLY; according to claim 1; wherein the drillable bottom has sealing means consisting of an annular grooving.

23) UNIT TO ADMINISTER INJECTABLE MEDICATION MANUALLY OR AUTOMATICALLY; according to claims 21 and 22; wherein the annular grooving forms ledges of rounded section.

24) UNIT TO ADMINISTER INJECTABLE MEDICATION MANUALLY OR AUTOMATICALLY; according to claims 21 and 22; wherein the annular grooving forms ledges of rectangular section.

25) UNIT TO ADMINISTER INJECTABLE MEDICATION MANUALLY OR AUTOMATICALLY; according to claims 21 and 22; wherein the annular grooving forms ledges of angular section, the sloping side of which is arranged so that it helps with the forward movement inside the tubular body.
